(19) United States
(12) Patent Application Publication
(10) Pub. No.: US 2026/0083413 A1
(43) Pub. Date: Mar. 26, 2026

(54) RADIOGRAPHIC IMAGING APPARATUS, RADIOGRAPHIC IMAGING ASSISTANCE METHOD, AND NON-TRANSITORY COMPUTER-READABLE RECORDING MEDIUM STORING RADIOGRAPHIC IMAGING ASSISTANCE PROGRAM

(71) Applicant: KONICA MINOLTA, INC., Tokyo (JP)

(72) Inventor: Sumiya NAGATSUKA, Tokyo (JP)

(73) Assignee: KONICA MINOLTA, INC., Tokyo (JP)

(21) Appl. No.: 19/331,518

(22) Filed: Sep. 17, 2025

(30) Foreign Application Priority Data
Sep. 20, 2024 (JP) 2024-164021

(51) Int. Cl. A61B 6/04; A61B 6/00

(52) U.S. Cl. CPC A61B 6/0492; A61B 6/5205

(57) ABSTRACT

A radiographic imaging apparatus includes: an imager that captures a radiographic image of a target part of a subject during a predetermined motion on a frame-by-frame basis; at least one hardware processor that determines, on the frame-by-frame basis, whether or not a skeleton of the target part is at a reference position for the captured radiographic image, and identifies, as a reference position image, the radiographic image when the skeleton of the target part is at the reference position; and a notifier that provides a notification based on whether or not the reference position image has been identified.

### CROSS REFERENCE TO RELATED APPLICATIONS

The entire disclosure of Japanese Patent Application No. 2024-164021 filed on Sep. 20, 2024 is incorporated herein by reference in its entirety.

### BACKGROUND

### Technological Field

The present invention relates to a radiographic imaging apparatus, a radiographic imaging assistance method, and a non-transitory computer-readable recording medium storing a radiographic imaging assistance program.

### Description of Related Art

In an imaging apparatus that captures a radiographic image, positioning of a target part of a patient with respect to an imaging direction (a main direction of radiation irradiation) is important for performing diagnosis based on the radiographic image. When the positioning of the target part with respect to the imaging direction is not appropriate, the capturing of the radiographic image is repeated, which increases the burden on the patient and the technician who operates the imaging apparatus.

Therefore, for example, the apparatus disclosed in Japanese Unexamined Patent Publication No. 2017-217227 performs imaging for the positioning operation before the main imaging, determines whether the measurement region is appropriate, from the captured fluoroscopic image, and when the measurement region is not appropriate, a notification is provided to a technician who operates the apparatus. Thus, the radiological technician can perform the positioning operation with reference to the fluoroscopic image, and can confirm whether the positioning is correct before the main imaging.

However, even if the apparatus disclosed in Japanese Unexamined Patent Publication No. 2017-217227 is used, when the measurement region is not appropriate, the technician needs to perform a positioning operation, and the burden is still not light. In particular, it is a great burden for an inexperienced technician to perform the positioning operation with reference to the fluoroscopic image.

### SUMMARY

An object of the present invention is to provide a radiographic imaging apparatus, a radiographic imaging assistance method, and a non-transitory computer-readable recording medium storing a radiographic imaging assistance program, which are capable of reducing the burden on a technician at the time of capturing a radiographic image.

In order to realize at least one of the above-described objects, a radiographic imaging apparatus reflecting one aspect of the present invention includes: an imager that captures a radiographic image of a target part of a subject during a predetermined motion on a frame-by-frame basis; at least one hardware processor that determines, on the frame-by-frame basis, whether or not a skeleton of the target part is at a reference position for the captured radiographic image, and identifies, as a reference position image, the radiographic image when the skeleton of the target part is at the reference position; and a notifier that provides a notification based on whether or not the reference position image has been identified.

In order to realize at least one of the above-described objects, a radiographic imaging assistance method reflecting one aspect of the present invention includes: capturing, by a radiographic imaging apparatus, a radiographic image of a target part of a subject during a predetermined motion on a frame-by-frame basis; determining, by the radiographic imaging apparatus, on the frame-by-frame basis, whether or not a skeleton of the target part is at a reference position for the captured radiographic image, and identifying, as a reference position image, the radiographic image when the skeleton of the target part is at the reference position; and providing, by the radiographic imaging apparatus, a notification based on whether or not the reference position image has been identified.

In order to realize at least one of the above-described objects, a non-transitory computer-readable recording medium storing a radiographic imaging assistance program that causes a computer of a radiographic imaging apparatus to perform: capturing a radiographic image of a target part of a subject during a predetermined motion on a frame-by-frame basis; determining, on the frame-by-frame basis, whether or not a skeleton of the target part is at a reference position for the captured radiographic image and identifying, as a reference position image, the radiographic image when the skeleton of the target part is at the reference position; and providing a notification based on whether or not the reference position image has been identified.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an explanatory diagram illustrating an example of a configuration of a radiographic image processing system according to an embodiment of the present invention;

FIG. 2 is a block diagram illustrating an example of a functional configuration of an imaging controller in a radiographic imaging apparatus constituting the radiographic image processing system;

FIG. 3 is a block diagram illustrating an example of a functional configuration of the radiographic imaging control apparatus constituting the radiographic image processing system;

FIG. 4 is a block diagram illustrating an example of a functional configuration of a radiographic image analysis apparatus constituting the radiographic image processing system;

FIG. 5 is a flowchart illustrating a radiographic imaging assistance method performed in the radiographic imaging apparatus;

FIG. 6 is a diagram illustrating an imaging direction, a reference position (determination condition), an operation, a recognition and measurement target, and a reference position image for each target part;

FIG. 7 is a diagram illustrating an example of a frame image in a maximum flexion state in a flexion-extension motion of the cervical spine;

FIG. 8 is a diagram illustrating an example of a frame image in a maximum extension state in the flexion extension motion of the cervical spine;

FIG. 9 is a schematic diagram illustrating an example of a frame image in a normal state in a ulnar-radial deviation motion of a wrist joint;

FIG. 10 is a schematic diagram illustrating an example of a frame image in a dynamic ulnocarpal abutment syndrome state in an ulnar-radial deviation motion of a wrist joint;

FIG. 11 is a diagram illustrating an example of a frame image indicating the position of a head of humerus with respect to a glenoid cavity of scapula in an abduction-elevation motion of a shoulder joint;

FIG. 12 is a diagram illustrating an example of a frame image indicating the position of a greater tubercle in an internal-external rotation motion of a shoulder joint;

FIG. 13 is a diagram illustrating an example of a frame image indicating the positions of a femoral head and a hip joint acetabulum in a femoral abduction motion;

FIG. 14 is a diagram illustrating an example of a frame image indicating the state of a knee joint in the loaded motion of the knee joint;

FIG. 15 is a diagram illustrating an example of a frame image indicating the state of the knee joint in an internal-external rotation motion of the knee joint;

FIG. 16 is a diagram illustrating an example of an imaging method in an axial direction of the knee joint;

FIG. 17A is a schematic diagram of a frame image obtained by imaging the knee joint in the axial direction and is an example of a reference position image;

FIG. 17B is a schematic diagram of a frame image obtained by imaging the knee joint in the axial direction, and is an example in which the bending angle of the knee is small and the frame image does not serve as the reference position image; and

FIG. 17C is a schematic diagram of a frame image obtained by imaging the knee joint in the axial direction, and is an example in which the bending angle of the knee is large and the frame image does not serve as the reference position image.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, embodiments of the present invention will be described in detail with reference to the drawings.

### Configuration of Radiographic Image Processing System

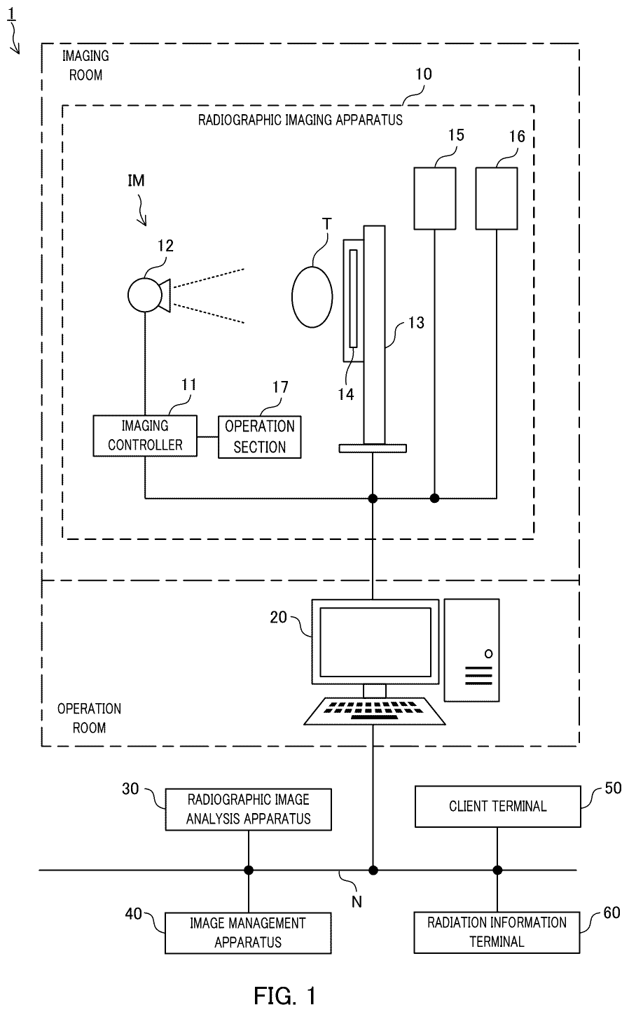

FIG. 1 is a diagram illustrating a radiographic image processing system 1 according to the present embodiment. The radiographic image processing system 1 includes a radiographic imaging apparatus 10, a radiographic imaging control apparatus (console apparatus) 20, a radiographic image analysis apparatus 30, an image management apparatus 40, and a client terminal 50.

In the example illustrated in FIG. 1, the radiographic imaging apparatus 10 is placed in an imaging room, and the radiographic imaging control apparatus 20 is placed in an operation room. The radiographic imaging apparatus 10, the radiographic imaging control apparatus 20, the radiographic image analysis apparatus 30, the image management apparatus 40, and the client terminal 50 are connected to each other via a communication network N. As the communication network N, for example, a network compliant with the Digital Image and Communications in Medicine (DICOM) standard or the like is used.

Furthermore, the communication network N is connected to a radiation information terminal 60 serving as a radiation information system that transmits information on a radiation inspection, for example, examination order information on a patient, to the radiographic image processing system 1. The radiation information terminal 60 is, for example, a radiology information system (RIS).

The radiographic imaging apparatus 10 performs radiographic dynamic imaging (hereinafter referred to as dynamic imaging), which is imaging of a dynamic image of a radiographic image, based on the control of the radiographic imaging control apparatus 20. In the present embodiment, as described later, the radiographic imaging apparatus 10 specifies, as a reference position image, a radiographic image at the reference position from a dynamic image including a plurality of radiographic images. The radiographic imaging control apparatus 20 controls the radiographic imaging apparatus 10 on the basis of the examination order information or the like transmitted from the radiation information terminal 60. Further, the radiographic imaging control apparatus 20 transmits the dynamic image generated by the radiographic imaging apparatus 10 or the specified reference position image to the radiographic image analysis apparatus 30 or the image management apparatus 40. The radiographic image analysis apparatus 30 performs dynamic analysis on the dynamic image, and transmits an analysis result of the dynamic analysis to the image management apparatus 40. The image management apparatus 40 as the medical image management system manages the transmitted dynamic image, reference position image, and analysis result. The image management apparatus 40 is, for example, a picture archiving and communication system (PACS). The client terminal 50 acquires the dynamic image, the reference position image, and the analysis result from the image management apparatus 40, and provides the dynamic image, the reference position image, and the analysis result to a medical worker such as a doctor in a browsable manner.

In the present embodiment, dynamic imaging refers to obtaining a plurality of frame images by repeatedly irradiating a subject with pulsed radiation (for example, X-rays) at a predetermined frame rate (pulse irradiation). The dynamic image refers to a series of frame images obtained by dynamic imaging. Furthermore, the dynamic analysis refers to analysis processing performed on the dynamic image, and includes, in addition to processing for analyzing the movement of the subject based on the dynamic image, processing for analyzing the dynamic image to emphasize or attenuate (remove) a predetermined structure.

The radiographic imaging apparatus 10, the radiographic imaging control apparatus 20, and the radiographic image analysis apparatus 30 each include a processor and a memory. Each of the radiographic imaging apparatus 10, the radiographic imaging control apparatus 20, and the radiographic image analysis apparatus 30 is a type of computer that realizes a predetermined function by reading, loading, and executing a program stored in a memory.

### Radiographic Imaging Apparatus 10

As illustrated in FIG. 1, the radiographic imaging apparatus 10 includes an imaging controller 11, a radiation irradiator 12, an imaging table 13, a radiation detector 14, a display section 15, a sound output section 16, and an operation section 17.

The imaging controller 11 acquires setting information regarding the setting of the dynamic imaging from the radiographic imaging control apparatus 20. The imaging controller 11 sets imaging conditions for performing dynamic imaging based on the setting information, controls the radiation irradiator 12 based on the imaging conditions to irradiate a patient (subject) with radiation, and performs imaging. The imaging controller 11 is, for example, a computer including one or more hardware processors, and includes a central processing unit (CPU), a random access memory (RAM), a read only memory (ROM), and the like. A radiographic imaging assistance program which will be described later is stored in a computer-readable non-transitory recording medium, and the storage section (not illustrated) of the imaging controller 11 stores the program from the recording medium.

The setting information is information on settings for performing dynamic imaging on a patient. The setting information is set by an operator of the radiographic image processing system 1, for example, an imaging technician, in the radiographic imaging control apparatus 20 to be described later.

The imaging conditions include, for example, various conditions such as a pulse rate, a pulse width, a pulse interval, the number of imaging frames per imaging, a dose per unit time of radiation irradiation, a motion of the target part T of the patient, and a respiratory state. The pulse rate is the number of times of radiation irradiation per second, and matches the frame rate of image data. The pulse width is a radiation irradiation time per radiation irradiation. The pulse interval is a time from the start of one radiation irradiation to the start of the next radiation irradiation, and matches with a time interval (frame interval) between a plurality of image data. The imaging conditions may be automatically determined by the imaging controller 11 of the radiographic imaging apparatus 10 based on the setting information.

The radiation irradiator 12 is disposed so as to face the radiation detector 14. The radiation irradiator 12 emits radiation under the control of the imaging controller 11.

The radiation detector 14 is composed of a semiconductor image sensor, such as a flat panel detector (FPD). The radiation detector 14 includes a substrate on which a plurality of detection elements (pixel) is arranged in a matrix. Each pixel of the substrate detects the radiation emitted from the radiation irradiator 12 according to its intensity, converts the detected radiation into an electrical signal, and accumulates the electrical signal. Each pixel of the substrate is provided with, for example, a switching section such as a thin film transistor (TFT).

The radiation detector 14 controls the switching section of each pixel based on image reading conditions input from the radiographic imaging control apparatus 20 to read the electrical signal accumulated in each pixel, and outputs intensity information of each pixel to the image generator 113. The image reading condition is, for example, a frame rate, a frame interval, a pixel size, an image size (matrix size), or the like. The frame rate is the number of frame images acquired per second, and matches the pulse rate. The frame interval is a time period from the start of one operation of acquiring image data to the start of the operation of acquiring the next frame image, and corresponds to the pulse interval.

The imaging controller 11 and the radiation detector 14 are connected to each other, and exchange synchronization signals with each other so as to synchronize the radiation irradiation operation and the image reading operation.

Note that although FIG. 1 illustrates, as an example, a configuration in which the radiation detector 14 is fixed to the imaging table 13, a wireless type radiation detector that can be freely disposed may be used as the radiation detector 14. In this case, the radiation detector 14 can be disposed at an appropriate position according to the target part of the patient.

In this way, in the radiographic imaging apparatus 10, the radiation irradiator 12 emits radiation under the control of the imaging controller 11. Then, the radiographic imaging apparatus 10 performs dynamic imaging of a radiographic image by the radiation detector 14 generating image data based on the intensity of the emitted radiation. In the present embodiment, the imaging controller 11, the radiation irradiator 12, and the radiation detector 14 correspond to an imager IM of the present invention. In the present embodiment, the imager IM captures a radiographic image of the target part T of the subject during the predetermined motion in units of frames.

The display section 15 and the sound output section 16 provide the patient with instructions on the predetermined motion of the target part T, the respiratory state, and/or the like when the dynamic imaging of the target part T of the patient is performed. The display section 15 is, for example, a display device such as a cathode ray tube (CRT), a liquid crystal display, or an organic electro luminescence (EL) display, and includes a display screen. The sound output section 16 is, for example, a sound output device such as a speaker. The sound output section 16 provides the patient with an instruction on the predetermined motion of the target part T, a respiratory state, and/or the like by, for example, automatic voice. Each of the display section 15 and the sound output section 16 may provide the instruction having the same content to the patient, or only one of them may provide the instruction.

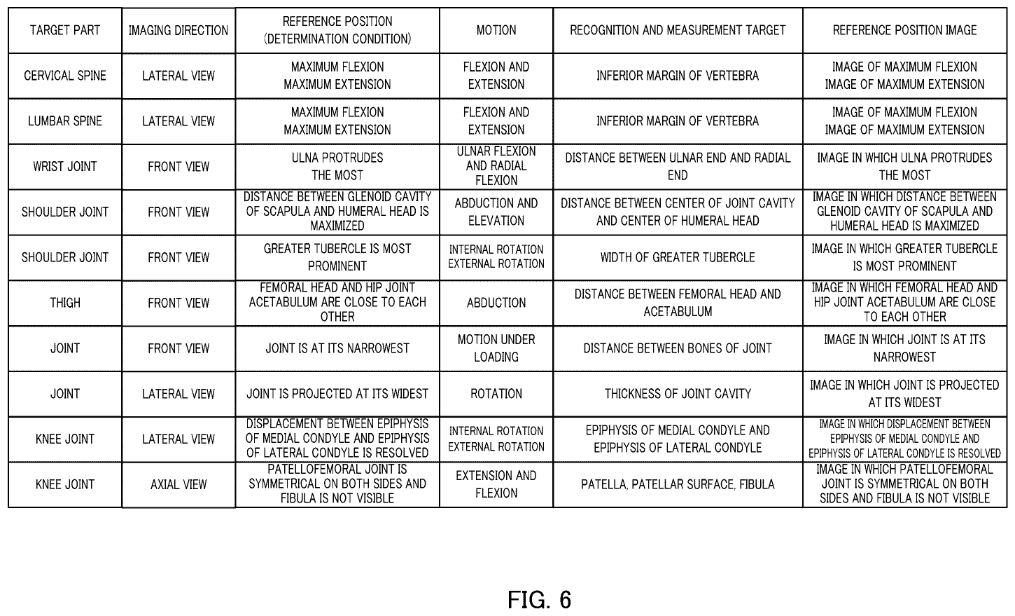

As the predetermined operation of the target part T, an operation corresponding to the target part T is illustrated in FIG. 6 described later. Any of the operations corresponding to the target part T is an operation including a state in which the skeleton of the target part T is positioned at the reference position during the operation.

The operation section 17 is an input device which is used when an operator operates the radiographic imaging apparatus 10 in an imaging room. The operation section 17 is, for example, a keyboard including cursor keys, number input keys, and various function keys, a pointing device such as a mouse or a trackball, and an operation device such as a touch screen. The operation section 17 generates an instruction signal based on an input by the operator and outputs the instruction signal to the imaging controller 11. The operation section 17 also includes an exposure switch (not illustrated). The exposure switch is used by the operator to instruct the radiographic imaging apparatus 10 to perform radiation irradiation and image capturing.

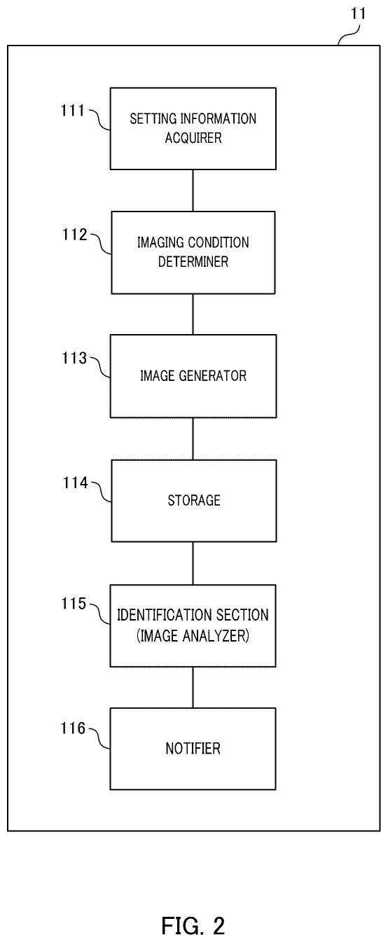

FIG. 2 is a block diagram illustrating an example of a functional configuration of the imaging controller 11 in the radiographic imaging apparatus 10 constituting the radiographic image processing system 1. The imaging controller 11 includes a setting information acquirer 111, an imaging condition determiner 112, an image generator 113, a storage 114, an identification section 115, and a notifier 116.

The setting information acquirer 111 acquires setting information from the radiographic imaging control apparatus 20.

The imaging condition determiner 112 determines imaging conditions for performing dynamic imaging of the patient, based on the setting information.

For example, in the case of screening, emergency, or the like, the imaging condition determiner 112 determines the imaging condition by causing the operator to select at least one imaging condition from a plurality of imaging conditions defined in advance. The imaging condition determiner 112 may cause the operator to select examination order information and determine the imaging conditions based on the selected examination order information.

The image generator 113 executes dynamic imaging on the target part T of the patient based on the determined imaging conditions, and generates a plurality of frames of radiographic image. More specifically, the image generator 113 controls the operations of the radiation irradiator 12 and the radiation detector 14 based on the imaging conditions, and generates image data by acquiring, for each pixel, intensity information relating to the intensity of radiation transmitted through the subject from the radiation detector 14. In this way, a radiographic image is captured.

The storage 114 stores information on imaging conditions and the like in advance.

During the imaging, the identification section 115 determines, on a frame-by-frame basis, whether the skeleton of the target part T is located at the reference position in the captured radiographic image. Then, the identification section 115 identifies, as a reference position image, the radiographic image when the skeleton of the target part T is located at the reference position. For example, the identification section 115 includes an image analyzer that analyzes the radiographic image in units of frames, determines whether the skeleton of the target part T is at the reference position based on the analysis result of the image analysis by the image analyzer, and identifies the reference position image. For example, the image analyzer recognizes and measures a target for the recognition and measurement shown in FIG. 6 (to be described later) by image analysis, determines whether the skeleton of the target part T is at the reference position based on the recognition and measurement results, and identifies the reference position image. At this time, the image analyzer may recognize and measure the target using, for example, a training model obtained by machine learning of the target.

The notifier 116 provides a notification on the basis of whether or not the reference position image has been identified. For example, using the sound output section 16, the notifier 116 outputs a sound when the skeleton of the target part T is at the reference position during the predetermined motion. Furthermore, the notifier 116 may use the sound output section 16 to increase the volume of the sound as the skeleton of the target part T approaches the reference position during the predetermined motion. Furthermore, using the display section 15, when the skeleton of the target part T is located at the reference position during the predetermined motion, the notifier 116 may display, on the display screen of the display section 15, information indicating that the skeleton of the target part T has been positioned at the reference position.

By including the identification section 115 and the notifier 116 described above, the radiographic imaging apparatus 10 also functions as a radiographic imaging assistance apparatus that assists the operator in capturing a radiographic image at the reference position (reference position image).

### Radiographic Imaging Control Apparatus 20

The radiographic imaging control apparatus 20 is, for example, a computer such as a personal computer (PC) or a workstation. The radiographic imaging control apparatus 20 may be a desktop computer as in the example illustrated in FIG. 1, or may be a portable computer such as a notebook computer or a tablet computer.

The radiographic imaging control apparatus 20 receives examination order information from the radiation information terminal 60 or the like and transmits it to the radiographic imaging apparatus 10 to control the dynamic imaging of the radiographic imaging apparatus 10.

The examination order information includes various kinds of information such as instruction information at the time of imaging, patient information, inspection information, imaging information, and data attributes. The inspection information includes information such as an inspection ID and an inspection target part. The examination order information is generated, for example, when a doctor or the like requests the radiographic image processing system 1 to perform dynamic imaging of a patient.

The radiographic imaging control apparatus 20 generates setting information based on an input from the operator. The operator performs an input operation for generating the setting information, for example, with reference to the content of the examination order information.

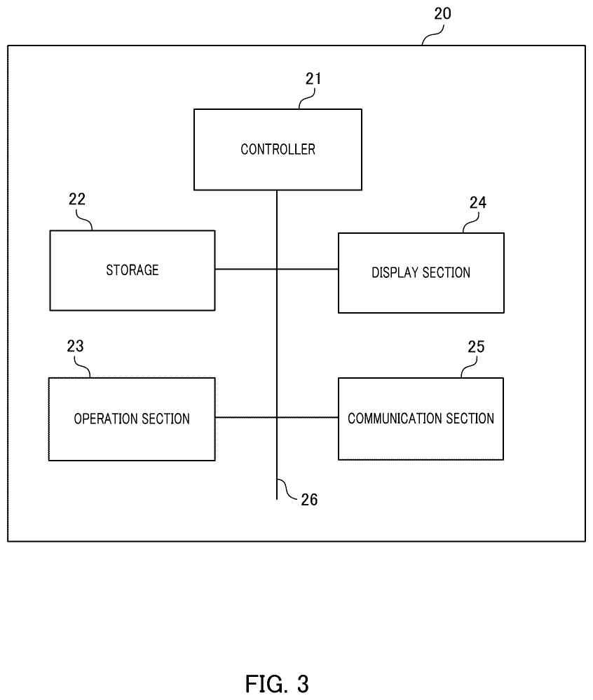

FIG. 3 is a block diagram illustrating an example of a functional configuration of the radiographic imaging control apparatus 20 constituting the radiographic image processing system 1. The radiographic imaging control apparatus 20 includes a controller 21, a storage 22, an operation section 23, a display section 24, and a communication section 25. The components of the radiographic imaging control apparatus 20 are connected to each other by a bus 26.

The radiographic imaging control apparatus 20 outputs, to the radiographic imaging apparatus 10, setting conditions set by an operator or the like and examination order information acquired in advance from the radiation information terminal 60 or the like, and controls imaging processing by the radiographic imaging apparatus 10. The radiographic imaging control apparatus 20 may display the dynamic image generated by the radiographic imaging apparatus 10, for example, for the operator to check.

The controller 21 includes a CPU, a RAM, and the like. In the controller 21, the CPU reads a system program and various processing programs stored in the storage 22 in response to an operation of the operation section 23, and loads the programs into the RAM. The CPU controls the operation of each unit of the radiographic imaging control apparatus 20 based on the loaded programs.

The storage 22 is constituted by a nonvolatile semiconductor memory, a hard disk, or the like. The storage 22 stores various programs executed by the controller 21, parameters necessary for execution of processing by the programs, or data such as processing results (dynamic image, reference position image, and/or the like). The various programs are stored in the form of readable program codes, and the controller 21 sequentially executes operations in accordance with the program codes.

Further, the storage 22 stores image reading conditions for performing dynamic imaging. Further, the storage 22 stores examination order information transmitted from the radiation information terminal 60 or the like. When the radiographic imaging control apparatus 20 controls the dynamic imaging of the radiographic imaging apparatus 10, the radiographic imaging control apparatus 20 reads out the image reading condition and the examination order information corresponding to the patient from the storage 22 and transmits them.

The operation section 23 is an operation device such as a keyboard including cursor keys, number input keys, and various function keys, a pointing device such as a mouse or a trackball, and a touch screen. The operation section 23 generates an instruction signal based on an input of an operator, and outputs the instruction signal to the controller 21.

The display section 24 is constituted by a display device such as a CRT, a liquid crystal display, or an organic EL display. The display section 24 displays an input instruction from the operation section 23, image data (dynamic image or the like) generated by the radiographic imaging apparatus 10, or the like in accordance with an instruction of a display signal input from the controller 21.

The communication section 25 transmits and receives data to and from the radiographic imaging apparatus 10, the radiographic image analysis apparatus 30, the radiation information terminal 60, and the like.

### Radiographic Image Analysis Apparatus 30

The radiographic image analysis apparatus 30 is, for example, a computer such as a PC or a workstation. The radiographic image analysis apparatus 30 may be a desktop computer or a portable computer such as a notebook computer or a tablet computer.

The radiographic image analysis apparatus 30 performs a dynamic analysis on the dynamic image captured by the radiographic imaging apparatus 10 based on the setting information set in the radiographic imaging control apparatus 20.

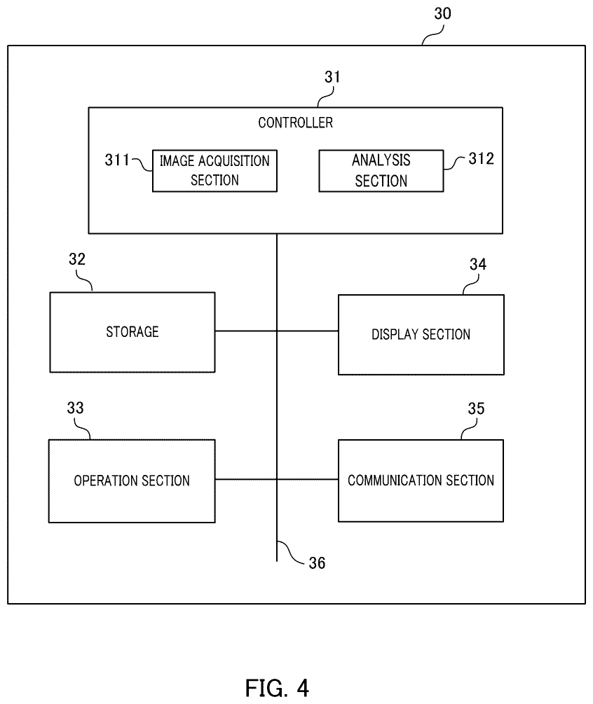

FIG. 4 is a block diagram illustrating an example of a functional configuration of the radiographic image analysis apparatus 30 constituting the radiographic image processing system 1. The radiographic image analysis apparatus 30 includes a controller 31, a storage 32, an operation section 33, a display section 34, and a communication section 35. These components of the radiographic image analysis apparatus 30 are connected to each other by a bus 36.

The controller 31 includes a CPU, a RAM, and the like. In the controller 31, in response to an operation of the operation section 33, the CPU reads a system program and various processing programs stored in the storage 32, and loads them into the RAM. Then, the CPU executes operation control, dynamic analysis, and the like of each unit of the radiographic image analysis apparatus 30 based on the loaded programs.

The controller 31 includes an image acquisition section 311 and an analysis section 312.

The image acquisition section 311 acquires a dynamic image which is a plurality of frames of radiographic images generated by the radiographic imaging apparatus 10.

The analysis section 312 performs the dynamic analysis set in the setting information on the dynamic image acquired from the radiographic imaging apparatus 10, and acquires an analysis result. At this time, in a case where the analysis section 312 cannot analyze the dynamic image (cannot acquire the analysis result), the analysis is determined to be impossible.

The analysis section 312 has, for example, an orthopedic measurement mode as the type of dynamic analysis.

The orthopedic measurement mode is, for example, a mode for measuring a positional change or the like of a designated bone in a limb or the like and displaying a movement trajectory or the like.

The storage 32 is constituted by a nonvolatile semiconductor memory, a hard disk, or the like. The storage 32 stores various programs to be executed by the controller 31, parameters required for execution of processing by the programs, or data such as processing results (e.g., dynamic image and analysis result). The various programs are stored in the form of readable program codes, and the controller 31 sequentially executes operations in accordance with the program codes.

In addition, the storage 32 stores patient information or inspection information related to each dynamic image generated by the radiographic imaging apparatus 10, and list information indicating a status (for example, a progress state such as during reception, during dynamic analysis, or analysis completion). Furthermore, analysis results and measurement results are stored in the storage 32 in association with the dynamic image.

The operation section 33 is an operation device such as a keyboard including cursor keys, number input keys, and various function keys, a pointing device such as a mouse or a trackball, and a touch screen. The operation section 33 generates an instruction signal based on an input by the operator, and outputs the instruction signal to the controller 31. The operation section 33 may include a touch screen on the display screen of the display section 34, and in this case, outputs an instruction signal input through the touch screen to the controller 31.

The display section 34 is constituted by a display device such as a CRT, a liquid crystal display, or an organic EL display. The display section 34 displays an input instruction from the operation section 33, image data (a dynamic image analysis result, a measurement result, and the like) generated by the radiographic imaging apparatus 10, and the like in accordance with an instruction of a display signal input from the controller 31.

The communication section 35 transmits and receives data to and from the radiographic imaging control apparatus 20, the image management apparatus 40, and the like.

### Radiographic Imaging Assistance Method

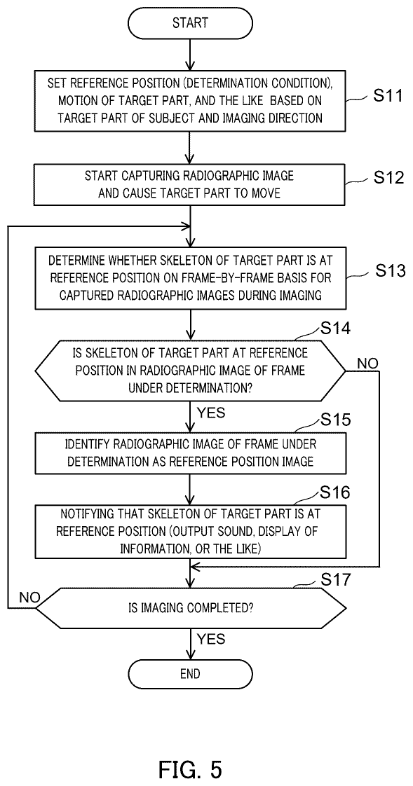

FIG. 5 is a flowchart illustrating a radiographic imaging assistance method performed in the radiographic imaging apparatus 10. A radiographic imaging assistance program for implementing the radiographic imaging assistance method described below is included in the programs executed by the imaging controller 11. FIG. 6 is a diagram illustrating an imaging direction, a reference position (determination condition), a motion, a recognition and measurement target, and a reference position image for each target part.

### (Step S11)

The imaging controller 11 of the radiographic imaging apparatus 10 sets the reference position (determination condition) of the skeleton of the target part, the motion of the target part, and the recognition and measurement target, on the basis of the target part of the patient (subject) and the imaging direction.

For example, the imaging controller 11 sets the target part and the imaging direction based on the examination order information including the information on the target part and the imaging direction transmitted from the radiation information terminal 60 or the like via the radiographic imaging control apparatus 20. Next, based on the target part and the imaging direction, the imaging controller 11 sets the reference position (determination condition) of the skeleton of the target part, the motion of the target part when determining whether the skeleton of the target part is at the reference position, and the recognition and measurement target to be used for the determination. Note that in a case where there is a plurality of motions of the target part and a plurality of reference positions (determination conditions) with respect to the set target part and imaging direction, the radiographic imaging apparatus 10 presents the plurality of motions and reference positions (determination conditions) on the display section 15 so that the operator can select.

As illustrated in FIG. 6, when the target part is the “cervical spine” and the imaging direction is a “lateral view,” the imaging controller 11 sets “maximum flexion” and “maximum extension” as the reference positions (determination condition), and sets “flexion and extension” as the motion. At this time, the imaging controller 11 sets the “inferior margin of vertebra” as a recognition target of the cervical spine in the radiographic image of each frame.

As illustrated in FIG. 6, the reference position (determination condition), the motion, and the recognition and measurement target are also set for each of the other target parts on the basis of the target part and the imaging direction. In a case where the target part is a joint such as a “shoulder joint” or a “knee joint,” a plurality of motions and reference positions (determination conditions) are set. Therefore, as described above, the reference position (determination condition) is presented together with the plurality of motions, and the operator is allowed to make a selection, thereby setting the reference position (determination condition), the motion, and the recognition and measurement target.

(step S12)

The radiographic imaging apparatus 10 starts the capturing of the radiographic image (dynamic image) and causes the patient to move the target part. For example, capturing of a radiographic image (a dynamic image) is started by an operator pressing an exposure switch. In addition, at the time of imaging, the imaging controller 11 instructs the patient to perform the motion of the target part by, for example, an automatic voice using the sound output section 16.

For example, when the target part is the “cervical spine” and the imaging direction is a “lateral view,” the imaging controller 11 instructs the patient to perform a flexion-extension motion of the cervical spine by an automatic voice using the sound output section 16 (see FIG. 6).

The same applies to the other target parts, and the imaging controller 11 instructs the patient to perform the motion corresponding to the target part and the imaging direction by an automatic voice using the sound output section 16 (see FIG. 6).

(step S13)

During imaging, the imaging controller 11 (identification section 115) determines, on a frame-by-frame basis, whether the skeleton of the target part is located at the reference position in the captured radiographic image.

For example, in a case where the target part is the “cervical spine” and the imaging direction is a “lateral view,” the reference positions (determination conditions) are “maximum flexion” and “maximum extension.” The identification section 115 performs determination on a frame-by-frame basis for a radiographic image obtained by causing the cervical spine to flex and extend during imaging, by the image analysis by the above-described image analyzer, the “inferior margin of vertebra” which is the recognition target is recognized, and it is determined whether the cervical spine is at the maximum flexion position or not the cervical spine is at the maximum extension position.

The same applies to the other target parts. The identification section 115 makes the determination on a frame-by-frame basis for the radiographic image in which the target part is moved during imaging, through the image analysis by the image analyzer, the recognition target or the like is recognized, and it is determined whether the skeleton of the target part is at the reference position.

As the imaging controller 11, one having a high information processing capability is used, so that the identification section 115 makes the determination in units of frames in real time during imaging.

### (Step S14)

If the skeleton of the target part is located at the reference position in the radiographic image of the frame under determination (YES), the imaging controller 11 (the identification section 115) proceeds to step S15, and if the skeleton of the target part is not located at the reference position (NO), the imaging controller 11 proceeds to step S17.

### (Step S15)

In a case in which it is determined in Step S14 that the skeleton of the target part is located at the reference position (YES), the imaging controller 11 (the identification section 115) identifies the radiographic image of the frame under determination as the reference position image.

### (Step S16)

The imaging controller 11 (notifier 116) provides a notification that the skeleton of the target part is at the reference position. The notifier 116 outputs a sound when the skeleton of the target part T is located at the reference position, for example, using the sound output section 16. Furthermore, the notifier 116 may use the sound output section 16 to increase the volume of the sound as the skeleton of the target part T approaches the reference position. Furthermore, for example, when the skeleton of the target part T is located at the reference position, the notifier 116 may display, using the display section 15, information indicating that the skeleton of the target part T has been located at the reference position on the display screen of the display section 15.

### (Step S17)

The imaging controller 11 confirms whether or not imaging has ended, and in the case where imaging has ended (YES), ends the series of processing, and in the case where imaging has not ended (NO), returns to step S13. That is, the imaging controller 11 repeats Steps S13 to S17 until imaging ends, to perform determination, identification, and the like on a frame-by-frame basis. The confirmation of whether the imaging has ended is performed by the imaging controller 11, for example, by confirming whether the operator is pressing the exposure switch.

As described above, the radiographic imaging apparatus 10 can capture a dynamic image (a plurality of radiographic images) of the target part and identify the reference position image. After capturing the dynamic image and identifying the reference position image, the radiographic imaging apparatus 10 transmits the dynamic image and the reference position image to the radiographic image analysis apparatus 30, the image management apparatus 40, or the like via the radiographic imaging control apparatus 20.

The radiographic image analysis apparatus 30 analyzes the dynamic image transmitted from the radiographic imaging control apparatus 20 in the orthopedic measurement mode, and as an analysis result, measures a change in the position of the designated bone or displays the trajectory of the movement.

Then, the analysis result analyzed by the radiographic image analysis apparatus 30 is transmitted to the image management apparatus 40 together with the dynamic image, and can be viewed by a doctor or the like from the client terminal 50, for example.

Since the doctor using the client terminal 50 can view the reference position image together with the dynamic image and the analysis result, the doctor can make a more accurate diagnosis without re-imaging.

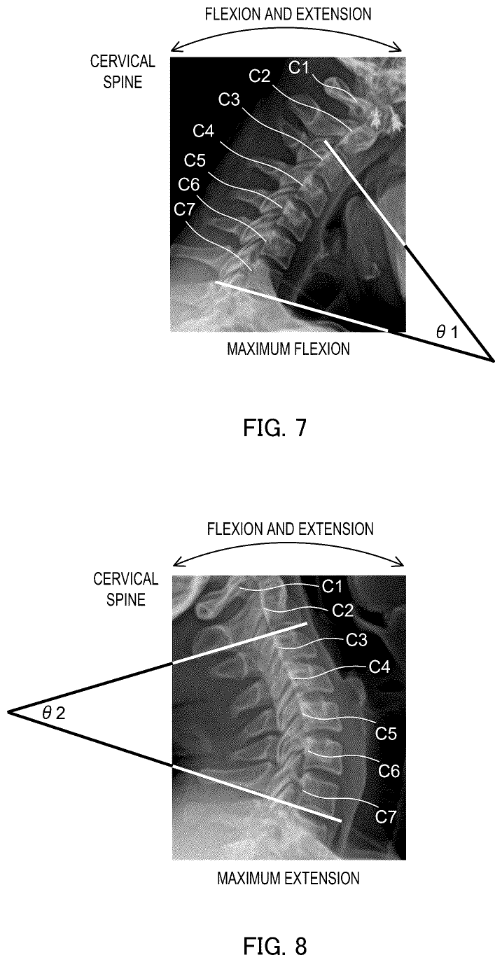

Here, a specific example of the above-described radiographic imaging assistance method will be described with reference to FIGS. 7 to 17C in addition to FIGS. 5 and 6.

### Specific Example 1—Cervical Spine

FIG. 7 is a diagram illustrating an example of a frame image in a state of maximum flexion in a flexion-extension motion of the cervical spine. FIG. 8 is a diagram illustrating an example of a frame image in a state of maximum extension in a flexion-extension motion of the cervical spine.

When diagnosing the function of the cervical spine, for example, as illustrated in FIG. 6, the imaging controller 11 sets the “cervical spine” as the target part and a “lateral view” as the imaging direction, based on the examination order information. Next, the imaging controller 11 sets “flexion and extension” as the motion, and “maximum flexion” and “maximum extension” as the reference positions and the determination conditions. Furthermore, as illustrated in FIG. 6, the imaging controller 11 sets the “inferior margin of vertebra” as a recognition target.

The radiographic imaging apparatus 10 causes the patient to flex and extend the cervical spine, performs dynamic imaging of the cervical spine during the flexion and extension of the cervical spine from the lateral view, and acquires a dynamic image. At this time, during imaging, the imaging controller 11 (identification section 115) determines, on a frame-by-frame basis, whether the cervical spine is at the position of the “maximum flexion” or “maximum extension”in the captured radiographic image.

For example, as illustrated in FIGS. 7 and 8, the identification section 115 recognizes the inferior margins of the cervical vertebra C2 and the cervical vertebra C7, and obtains, for each frame, angles θ1 and θ2 at which extended lines of the inferior margins of the cervical vertebrae intersect each other. The identification section 115 has determination angle information on the angles θ1 and θ2 in a case where the cervical spine is at the position of “maximum flexion” and “maximum extension” in advance, and compares the angles θ1 and θ2 obtained in units of frames with the determination angle information of the angles θ1 and θ2. The identification section 115 determines whether the cervical spine is at the position of “maximum flexion” or “maximum extension” based on the comparison result. Then, when determining that the cervical spine is in the “maximum flexion” position, the identification section 115 identifies the radiographic image of the frame under the determination as the reference position image. In addition, in a case where it is determined that the cervical spine is located at the “maximum extension” position, the identification section 115 identifies the radiographic image of the frame under the determination as the reference position image.

As described above, the identification section 115 compares the angles θ1 and θ2 obtained in units of frames with the determination angle information on the angles θ1 and θ2. Therefore, based on the comparison result, for example, the difference between the compared angles, the notifier 116 can provide a notification that the cervical spine is in the position of “maximum flexion” or “maximum extension” by sound or information display.

As described above, the radiographic imaging apparatus 10 can determine whether or not the cervical spine is in the “maximum flexion” position or the “maximum extension” position during the capturing of the lateral view of the cervical spine, and can identify the reference position image when it is determined that the cervical spine is in the “maximum flexion” position or the “maximum extension” position. As a result, it is possible to reduce the burden on the operator at the time of capturing a radiographic image.

### Specific Example 2—Lumbar Spine

The cervical spine has been described in the specific example 1, and the same processing as that for the cervical spine can be performed for the lumbar spine as the radiographic imaging assistance method. Accordingly, it is possible to determine whether or not the lumbar spine are in the “maximum flexion” position or the “maximum extension” position during imaging of the lateral view of the lumbar vertebra, and it is possible to identify the reference position image in a case where it is determined that the lumbar vertebra are in the “maximum flexion” position or the “maximum extension” position. As a result, it is possible to reduce the burden on the operator at the time of capturing a radiographic image.

### Specific Example 3—Wrist Joint

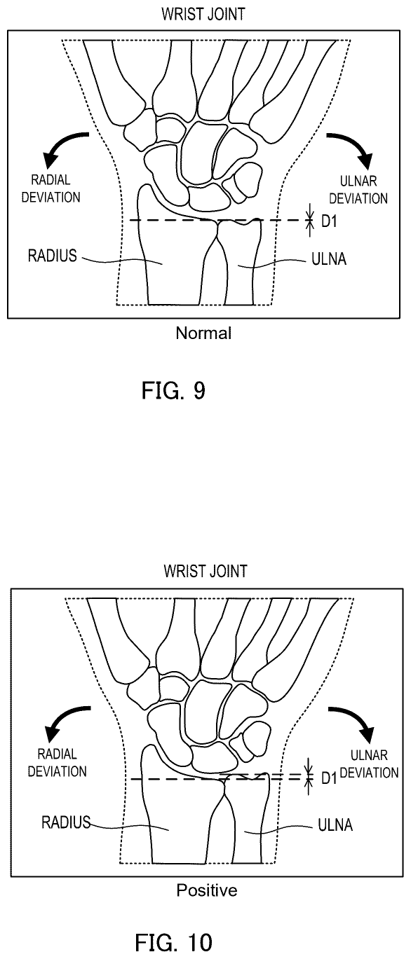

FIG. 9 is a schematic diagram illustrating an example of a frame image in a normal state in an ulnar-radial deviation motion of a wrist joint. FIG. 10 is a schematic diagram illustrating an example of a frame image in a state of dynamic ulnocarpal abutment syndrome in an ulnar-radial deviation motion of a wrist joint.

When the function of the wrist joint is diagnosed, the imaging controller 11 sets the “wrist joint” as the target part and the “front view” as the imaging direction, for example, based on the examination order information, as illustrated in FIG. 6. Then, the imaging controller 11 sets “ulnar flexion and radial flexion” as the motion, and “a state in which the ulna protrudes the most” as the reference position and the determination condition. Further, as shown in FIG. 6, the imaging controller 11 sets “the distance between the ulnar end and the radial end” as a measurement target.

Here, “ulnar deviation” is a motion of bending a joint of a wrist in a direction of the little finger (in a direction toward the ulna), and “radial deviation” is a motion of bending the joint of the wrist in a direction of the thumb (in a direction toward the radius).

The radiographic imaging apparatus 10 causes the patient to perform ulnar deviation and radial deviation of a wrist joint, performs dynamic imaging of the wrist joint during the ulnar deviation and radial deviation from the front view, and acquires a dynamic image. At this time, the imaging controller 11 (identification section 115) determines, on a frame-by-frame basis, whether the ulna is in the “state in which the ulna protrudes the most” in the captured radiographic image during the imaging.

For example, as illustrated in FIGS. 9 and 10, the identification section 115 recognizes the ulnar end and the radial end, and obtains the distance between the ulnar end and the radial end, for example, the distance D1 between the ulnar end and the radial end, for each frame. The identification section 115 has determination distance information on the distance D1 in the case of the “state in which the ulna protrudes the most” in advance, and compares the distance D1 obtained for each frame with the determination distance information of the distance D1. The identification section 115 determines, based on the result of the comparison, whether the ulna is in the “state in which the ulna protrudes the most.” Then, when it is determined that “the ulna protrudes the most,” the identification section 115 identifies the radiographic image of the frame during the determination as the reference position image.

As described above, the identification section 115 compares the distance D1 obtained for each frame with the determination distance information on the distance D1. Therefore, the notifier 116 can provide a notification that “the ulna protrudes the most” by sound or information display on the basis of the comparison result, for example, the difference between the compared distances.

As described above, the radiographic imaging apparatus 10 can determine whether or not the ulna is in the “state in which the ulna protrudes the most” during the imaging of the front view of the wrist joint, and when determining that “the ulna protrudes the most,” can identify the reference position image. Therefore, it is possible to reduce the burden on the operator at the time of capturing a radiographic image.

### Specific Example 4—Shoulder Joint

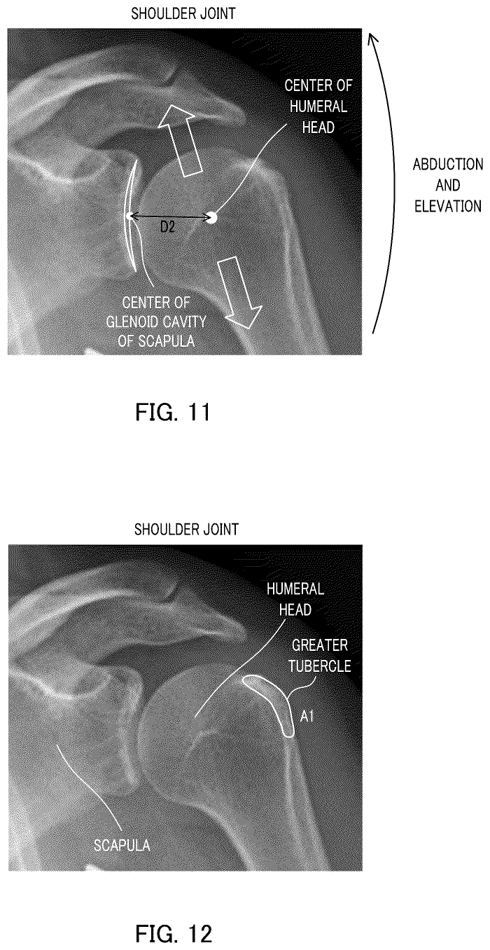

FIG. 11 is a diagram illustrating an example of a frame image indicating a position of a humeral head with respect to a glenoid cavity of a scapula in an abduction-elevation motion of a shoulder joint.

When diagnosing the function of the shoulder joint, the imaging controller 11 sets, for example, the “shoulder joint” as the target part and a “front view” as the imaging direction based on the examination order information, as illustrated in FIG. 6. Then, the imaging controller 11 sets “abduction-elevation” as the motion, and sets a “state in which the distance between the glenoid cavity of the scapula and the humeral head is maximized” as the reference position and the determination condition. Further, as shown in FIG. 6, the imaging controller 11 sets the “distance between the center of the glenoid cavity and the center of the humeral head” as a measurement target.

Here, the “abduction” is a motion of moving away from the median plane of the body, and here, a motion of separating the arm from the median plane of the body. Further, the “elevation” is a motion of raising the arm in the upward direction at the shoulder joint. The “abduction-elevation” is a motion of separating the arm from the median plane of the body and raising the arm in the upward direction.

The radiographic imaging apparatus 10 causes the patient to abduct and elevate his/her shoulder, performs dynamic imaging of the shoulder joint during the elevation-abduction from the front view, and acquires a dynamic image. At this time, during the imaging, the imaging controller 11 (identification section 115) determines, for each frame, whether or not the “the distance between the glenoid cavity of the scapula and the humeral head is maximized” in the captured radiographic image.

For example, as illustrated in FIG. 11, the identification section 115 recognizes the center of the glenoid cavity of the scapula and the center of the humeral head, and obtains, for each frame, the distance D2 between the center of the glenoid cavity of the scapula and the center of the humeral head. The identification section 115 has, in advance, determination distance information on the distance D2 in the case of the “state in which the distance between the glenoid cavity of the scapula and the humeral head is maximized,” and compares the distance D2 calculated for each frame with the determination distance information on the distance D2. Based on the result of the comparison, the identification section 115 determines whether or not “the distance between the glenoid cavity of the scapula and the humeral head is maximized.” When it is determined that “the distance between the glenoid cavity of the scapula and the humeral head is maximized,” the identification section 115 identifies the radiographic image of the frame during the determination as the reference position image.

As described above, the identification section 115 compares the distance D2 obtained for each frame with the determination distance information on the distance D2. Therefore, the notifier 116 can notify that “the distance between the glenoid cavity of the scapula and the humeral head is maximized” by sound or information display on the basis of the comparison result, for example, the difference between the compared distances.

As described above, the radiographic imaging apparatus 10 can determine whether or not “the distance between the glenoid cavity of the scapula and the humeral head is maximized” during the capturing of the front view of the shoulder joint. Then, in the case where it is determined that “the distance between the glenoid cavity of the scapula and the humeral head is maximized,” the radiographic imaging apparatus 10 can identify the reference position image. As a result, it is possible to reduce the burden on the operator at the time of capturing a radiographic image.

### Specific Example 5—Shoulder Joint

FIG. 12 is a diagram illustrating an example of a frame image indicating a position of a greater tubercle in an internal-external rotation motion of a shoulder joint.

When diagnosing the function of the shoulder joint, the imaging controller 11 sets, for example, a “shoulder joint” as the target part and a “front view” as the imaging direction based on the examination order information, as shown in FIG. 6. Then, the imaging controller 11 sets an “internal-external rotation” as the motion, and sets a “state in which the greater tubercle is most prominent” as the reference position and the determination condition. Furthermore, as illustrated in FIG. 6, the imaging controller 11 sets the “area of the greater tubercle” as a measurement target.

Here, the “internal rotation” is a movement of moving the shoulder joint closer to a median plane of the body (a movement of rotating inward), and “external rotation” is a movement of moving the shoulder joint away from the median plane (a movement of rotating outward).

The radiographic imaging apparatus 10 causes the patient to internally and externally rotate the shoulder joint, performs dynamic imaging of the shoulder joint during internal and external rotation from the front view, and acquires a dynamic image. At this time, the imaging controller 11 (identification section 115) determines, for each frame, whether the “the greater tubercle is most prominent” in the captured radiographic image during the imaging.

For example, as illustrated in FIG. 12, the identification section 115 recognizes the greater tubercle and obtains the area A1 of the greater tubercle on a frame-by-frame basis. The identification section 115 has determination area information on the area A1 in the case of the “state in which the greater tubercle is most prominent” in advance, and compares the area A1 obtained for each frame with the determination area information of the area A1. The identification section 115 determines, based on the result of the comparison, whether “the greater tubercle is most prominent.” Then, when determining that “the greater tubercle is most prominent,” the identification section 115 identifies the radiographic image of the frame under the determination as the reference position image.

As described above, the identification section 115 compares the area A1 calculated on a frame-by-frame basis with the determination area information on the area A1. Therefore, the notifier 116 can provide a notification that “the greater tubercle is most prominent” by sound or information display on the basis of the comparison result, for example, the difference between the compared areas.

As described above, the radiographic imaging apparatus 10 can determine whether or not “the greater tubercle is most prominent” during the imaging of the front view of the shoulder joint, and can identify the reference position image when it is determined that “the greater tubercle is most prominent.” As a result, it is possible to reduce the burden on the operator at the time of capturing a radiographic image.

### Specific Example 6—Femur

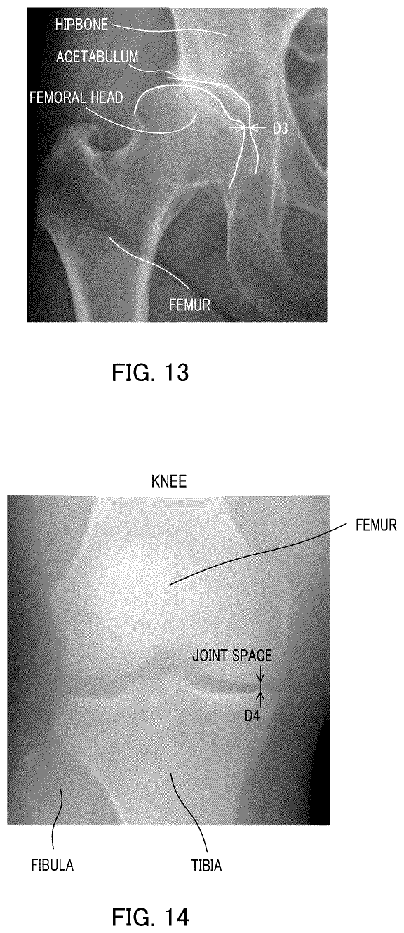

FIG. 13 is a diagram illustrating an example of a frame image indicating positions of a femoral head and a hip joint acetabulum in an abduction motion of the femur.

When diagnosing the function of the femur, the imaging controller 11 sets the “femur” as the target part and a “front view” as the imaging direction based on the examination order information, for example, as shown in FIG. 6. Then, the imaging controller 11 sets “abduction” as the motion, and sets a “state in which the femoral head and the hip joint acetabulum are close to each other” as the reference position and the determination condition. Furthermore, as illustrated in FIG. 6, the imaging controller 11 sets a “distance between the femoral head and the acetabulum” as a measurement target.

Here, the “abduction” is a motion of moving away from the median plane of the body, and here, is a motion of opening the foot outward at the hip joint.

The radiographic imaging apparatus 10 causes the patient to abduct his/her femur, performs dynamic imaging of the femur during the abduction from the front view, and acquires a dynamic image. At this time, during the imaging, the imaging controller 11 (identification section 115) determines, for each frame, whether or not “the femoral head and the hip joint acetabulum are close to each other” in the captured radiographic image.

For example, as illustrated in FIG. 13, the identification section 115 recognizes the femoral head and the hip joint acetabulum, and obtains distance D3 between the femoral head and the acetabulum for each frame. Here, as the distance D3, the distance of the portion at which the distance between the femoral head and the acetabulum is the narrowest is adopted.

The identification section 115 preliminarily has determination distance information on the distance D3 in the case of the “state in which the femoral head and the hip joint acetabulum are close to each other,” and compares the distance D3 calculated on a frame-by-frame basis with the determination distance information on the distance D3. The identification section 115 determines, based on the result of the comparison, whether “the femoral head and the hip joint acetabulum are close to each other.” Then, when determining that “the femoral head and the hip joint acetabulum are close to each other,” the identification section 115 identifies the radiographic image of the frame under the determination as the reference position image.

As described above, the identification section 115 compares the distance D3 obtained for each frame with the determination distance information on the distance D3. Therefore, the notifier 116 can provide a notification that “the femoral head and the hip joint acetabulum are close to each other” by sound or information display, based on the comparison result, for example, the difference between the compared distances.

As described above, the radiographic imaging apparatus 10 can determine whether or not “the femoral head and the hip joint acetabulum are close to each other” during the imaging of the front view of the femur, and can identify the reference position image in a case where it is determined that “the femoral head and the hip joint acetabulum are close to each other.” As a result, it is possible to reduce the burden on the operator at the time of capturing a radiographic image.

### Specific Example 7—Joint

Here, a knee joint will be described as an example of a joint. FIG. 14 is a diagram illustrating an example of a frame image indicating the state of a knee joint in the loaded motion of the knee joint.

When diagnosing the function of the knee joint, for example, the imaging controller 11 sets the “knee joint” as the target part and the “front view” as the imaging direction based on the examination order information. Then, the imaging controller 11 sets “extension and flexion under loading” as the motion, and sets a “state in which the knee joint is at its narrowest” as the reference position and the determination condition. Furthermore, the imaging controller 11 sets a “distance between the bones of the knee joint” as a measurement target.

Here, the “extension” refers to a motion of stretching a knee joint, and the “flexion” refers to a motion of flexing a knee joint. In addition, the “extension and flexion under loading” refers to a motion of extending and flexing the knee joint while a load is applied to the knee joint, for example, while a patient holds a heavy object.

The radiographic imaging apparatus 10 causes the patent to extend and flex the knee joint while the patent holds a heavy object, and performs dynamic imaging of the knee joint from the front view during the extension and flexion to acquire a dynamic image. At this time, the imaging controller 11 (identification section 115) determines, for each frame, whether or not “the knee joint is at its narrowest” in the captured radiographic image during the imaging.

For example, as illustrated in FIG. 14, the identification section 115 recognizes the femur and the tibia in the knee joint and obtains, for each frame, the distance D4 of the joint space between the femur and the tibia. Here, as the distance D4, the distance of the portion at which the distance between the femur and the tibia is the narrowest is adopted.

The identification section 115 holds in advance determination distance information on the distance D4 in the case where “the knee joint is at its narrowest,” and compares the distance D4 calculated for each frame with the determination distance information on the distance D4. The identification section 115 determines, based on the result of the comparison, whether or not “the knee joint is at its narrowest.” Then, when determining that “the knee joint is at its narrowest,” the identification section 115 identifies the radiographic image of the frame under the determination as the reference position image.

As described above, the identification section 115 compares the distance D4 obtained for each frame with the determination distance information on the distance D4. Therefore, the notifier 116 can provide a notification that “the knee joint is at its narrowest” by sound or information display on the basis of the comparison result, for example, the difference between the compared distances.

As described above, the radiographic imaging apparatus 10 can determine whether or not “the knee joint is at its narrowest” during the imaging of the front view of the knee joint, and can identify the reference position image when determining that “the knee joint is at its narrowest.” As a result, it is possible to reduce the burden on the operator at the time of capturing a radiographic image.

### Specific Example 8—Joint

A knee joint will also be described as an example of the joint.

When diagnosing the function of the knee joint, the imaging controller 11 sets, for example, the “knee joint” as the target part and the “lateral view” as the imaging direction based on the examination order information. Then, the imaging controller 11 sets “extension and flexion” as the rotational motion of the knee joint, and sets a “state in which the knee joint is most widely projected” as the reference position and the determination condition. The imaging controller 11 also sets the “thickness of the knee joint cavity” as a measurement target.

The radiographic imaging apparatus 10 causes the patient to extend and flex the knee joint, performs dynamic imaging of the knee joint during extension and flexion from the lateral view, and acquires a dynamic image. At this time, during the imaging, the imaging controller 11 (identification section 115) determines, for each frame, whether or not “the knee joint is most widely projected” in the captured radiographic image.

For example, the identification section 115 recognizes the knee joint cavity and obtains the thickness of the knee joint cavity for each frame. The identification section 115 holds, in advance, determination thickness information on the thickness of the knee joint cavity in the case where “the knee joint is most widely projected,” and compares the thickness of the knee joint cavity obtained on a frame-by-frame basis with the determination thickness information. The identification section 115 determines, based on the result of the comparison, whether or not the “the knee joint is most widely projected.” Then, in the case that the identification section 115 determines that “the knee joint is most widely projected,” the identification section 115 identifies the radiographic image of the frame under the determination as the reference position image.

As described above, the identification section 115 compares the thickness of the knee joint cavity obtained for each frame with the determination thickness information. Therefore, the notifier 116 can provide a notification that “the knee joint is most widely projected” by sound or information display on the basis of the comparison result, for example, the difference between the compared thicknesses.

As described above, the radiographic imaging apparatus 10 can determine whether or not “the knee joint is most widely projected” during the imaging of the lateral view of the knee joint, and can identify the reference position image when determining that “the knee joint is most widely projected.” As a result, it is possible to reduce the burden on the operator at the time of capturing a radiographic image.

### Specific Example 9—Knee Joint

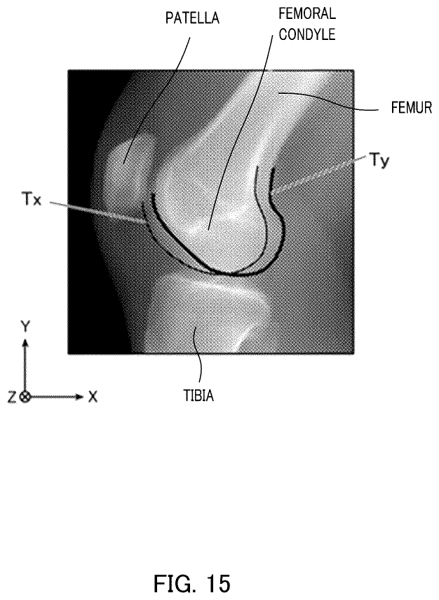

FIG. 15 is a diagram illustrating an example of a frame image indicating the state of the knee joint in an internal-external rotation motion of the knee joint. FIG. 15 is a frame image of the knee joint of the right knee captured laterally.

When diagnosing the function of the knee joint, the imaging controller 11 sets, for example, the “knee joint” as the target part and a “lateral view” as the imaging direction based on the examination order information. Next, the imaging controller 11 sets an “internal-external rotation” as the motion, and sets a “state in which the displacement between the epiphysis of the medial condyle and the epiphysis of the lateral condyle of the femur is resolved” as the reference position and the determination condition. That is, the state in which the epiphysis of the medial condyle and the epiphysis of the lateral condyle of the femur are displaced is a state in which the positioning of the knee joint is incorrect. Therefore, the imaging controller 11 sets the “epiphysis of the medial condyle and the epiphysis of the lateral condyle of the femur” as targets to be recognized.

The radiographic imaging apparatus 10 causes the patient to internally and externally rotate the knee joint, performs dynamic imaging of the knee joint during internal and external rotation from the lateral view, and acquires a dynamic image. At this time, during the imaging, the imaging controller 11 (the identification section 115) determines, for each frame, whether or not “the displacement between the epiphysis of the medial condyle and the epiphysis of the lateral condyle of the femur is resolved” in the captured radiographic image.

For example, as illustrated in FIG. 15, the identification section 115 recognizes the epiphysis Tx of the medial condyle and the epiphysis Ty of the lateral condyle in the knee joint, and obtains the displacement between the epiphysis Tx and the epiphysis Ty for each frame. When the epiphysis Tx and the epiphysis Ty are displaced, as shown in FIG. 15, the line indicating the epiphysis Tx and the epiphysis Ty is indicated by two lines. Note that in FIG. 15, the horizontal direction (left-right direction) of the radiographic image is defined as an X direction, the vertical direction (up-down direction) of the radiographic image is defined as a Y direction, and the imaging direction (radiation irradiation direction), which is a direction orthogonal to the X direction and the Y direction, is defined as a Z direction.

The identification section 115 obtains the displacement between the epiphysis Tx and the epiphysis Ty. The identification section 115 determines, based on the displacement between the epiphysis Tx and the epiphysis Ty, whether or not “the displacement between the epiphysis of the medial condyle and the epiphysis of the lateral condyle of the femur is resolved.” Then, when the displacement between the epiphysis Tx and the epiphysis Ty is less than a predetermined value, the identification section 115 determines that “the displacement between the epiphysis of the medial condyle and the epiphysis of the lateral condyle of the femur is resolved,” and in this case, identifies the radiographic image of the frame under the determination as the reference position image.

As described above, the identification section 115 obtains the displacement between the epiphysis Tx and the epiphysis Ty for each frame. Therefore, the notifier 116 can provide a notification that “the displacement between the epiphysis of the medial condyle and the epiphysis of the lateral condyle of the femur is resolved” by sound or information display on the basis of the calculation of the displacement between the epiphysis Tx and the epiphysis Ty.

As described above, the radiographic imaging apparatus 10 can determine whether or not “the displacement between the epiphysis of the medial condyle and the epiphysis of the lateral condyle of the femur is resolved” during the imaging of the lateral view of the knee joint. Then, in a case where it is determined that “the displacement between the epiphysis of the medial condyle and the epiphysis of the lateral condyle of the femur is resolved,” the radiographic imaging apparatus 10 can identify the reference position image. As a result, it is possible to reduce the burden on the operator at the time of capturing a radiographic image.

The reference position image of the lateral view of the knee joint captured as described above is useful for diagnosing knee osteoarthritis, rheumatoid arthritis, bone fracture, ligament damage, a primary tumor, a metastatic tumor, and the like.

### Specific Example 10—Knee Joint

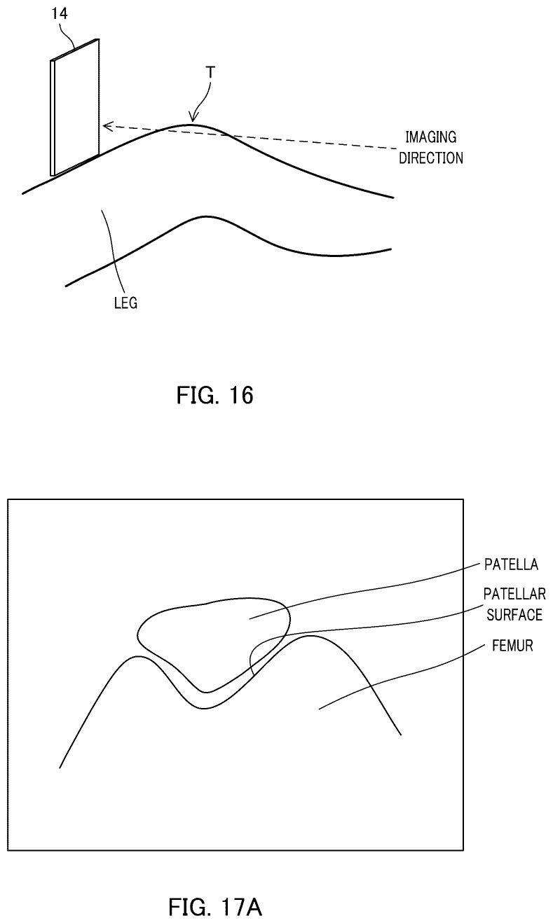

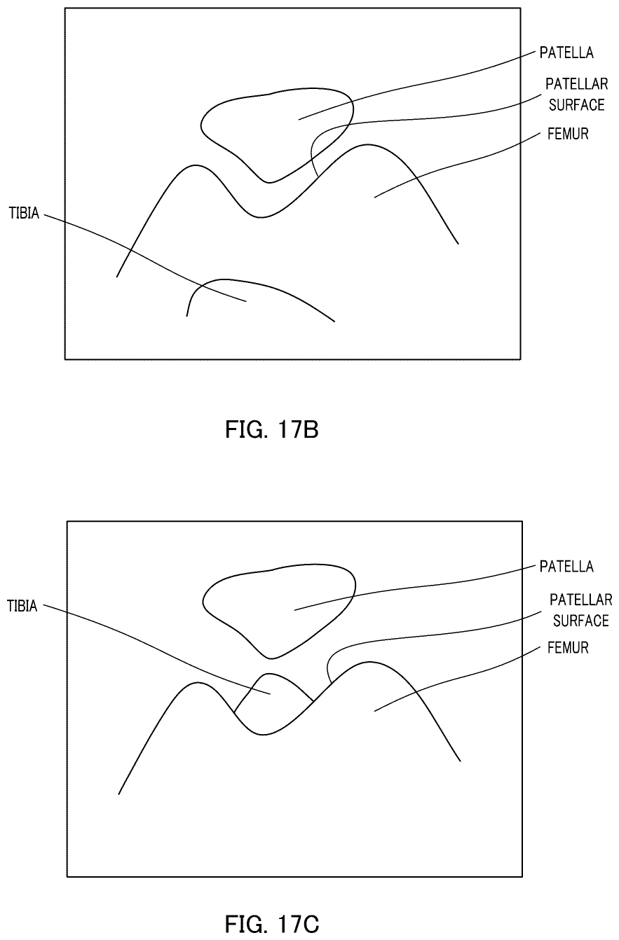

FIG. 16 is a diagram illustrating an example of an imaging method in an axial direction of a knee joint. Here, the Laurin imaging method is shown as an example, but another imaging method can be applied to the present invention. Furthermore, FIGS. 17A to 17C are schematic diagrams of frame images obtained by imaging the knee joint from the axial direction. FIG. 17A illustrates an example of a reference position image. FIG. 17B illustrates an example of an image in which the bending angle of the knees is small and which cannot serve as the reference position image. FIG. 17C illustrates an example of an image in which the bending angle of the knees is large and which cannot serve as the reference position image.

When diagnosing the function of the knee joint, the imaging controller 11 sets, for example, the “knee joint” as the target part and the “axial view” as the imaging direction based on the examination order information. Then, the imaging controller 11 sets “extension and flexion” as the motion, and sets a “state in which the patellofemoral joint is symmetrical on both sides and the fibula is not visible” as the reference position and the determination condition. The imaging controller 11 sets “the patella, the patellar surface of the femur, and the fibula” as targets to be recognized.

In the case of the Laurin imaging method, as shown in FIG. 16, the patient bends his/her knee in a sitting position. Then, as shown in FIG. 16, the radiation detector 14 is placed on the femur side, and radiation is irradiated to the patella in the caudocranial direction, which serves as the imaging direction.

Thereafter, the radiographic imaging apparatus 10 causes the patient to extend and flex the knee joint, performs dynamic imaging of the knee joint during extension and flexion from an axial view, and acquires a dynamic image. At this time, during the imaging, the imaging controller 11 (identification section 115) determines, for each frame, whether or not “the patellofemoral joint is symmetrical on both sides and the fibula is not visible” for the captured radiographic image.

For example, as illustrated in the FIGS. 17B and 17C, the identification section 115 recognizes the patella, the patellar surface of the femur, and the fibula in the knee joint, and determines, for each frame, whether the patellofemoral joint is symmetrical on both sides and whether the fibula is not visible.

The identification section 115 determines, based on the result of the above determination, whether or not “the patellofemoral joint is symmetrical on both sides and the fibula is not visible.” When it is determined that the “the patellofemoral joint is symmetrical on both sides and the fibula is not visible,” the identification section 115 identifies the radiographic image of the frame during the determination as the reference position image.

For example, the state of the knee joint shown in the schematic diagram of FIG. 17A is a state in which the patellofemoral joint is substantially symmetrical on both sides and the fibula is not visible. In this case, it is possible to determine that the “the patellofemoral joint is symmetrical on both sides and the fibula is not visible,” and in this case, the radiographic image of the frame during the determination is identified as the reference position image.

On the other hand, in the state of the knee joint illustrated in the schematic diagram of FIG. 17B, the patellofemoral joint is symmetrical on both sides, but the bending angle of the knee is small, and thus the fibula is visible below the patellar surface. In this case, it is not determined that “the patellofemoral joint is symmetrical on both sides and the fibula is not visible.”

In addition, in the state of the knee joint illustrated in the schematic diagram of FIG. 17C, the patellofemoral joint is substantially symmetrical on both sides, but the bending angle of the knee is large, and thus the fibula is visible above the patellar surface. In this case as well, it is not determined that the “the patellofemoral joint is symmetrical on both sides and the fibula is not visible.”

Here, the identification section 115 determines, for each frame, whether the patellofemoral joint is symmetrical on both sides and the fibula is not visible. Based on the determination, the notifier 116 provides a notification that “the patellofemoral joint is symmetrical on both sides and the fibula is not visible” by sound or information display.

As described above, the radiographic imaging apparatus 10 can determine whether or not the “the patellofemoral joint is symmetrical on both sides and the fibula is not visible” during imaging of the knee joint from an axial view. Then, in a case where it is determined that “the patellofemoral joint is symmetrical on both sides and the fibula is not visible,” the radiographic imaging apparatus 10 can identify the reference position image. As a result, it is possible to reduce the burden on the operator at the time of capturing a radiographic image.

The reference position image of the knee joint from an axial view captured as described above is useful for diagnosis of knee osteoarthritis, recurrent patellar dislocation, patellar fracture, intra-articular loose bodies, and the like.

### Summary

As described above, the radiographic imaging apparatus 10 includes an imager IM, an identification section 115, and a notifier 116. The imager IM captures, for each frame, a radiographic image of the target part T of the subject in the predetermined motion. During the imaging, the identification section 115 determines, on a frame-by-frame basis, whether the skeleton of the target part T is located at the reference position in the captured radiographic image. Then, the identification section 115 identifies, as the reference position image, the radiographic image when the skeleton of the target part T is located at the reference position. The notifier 116 provides a notification on the basis of whether or not the reference position image has been identified.

Since the radiographic imaging apparatus 10 notifies whether or not the reference position image has been identified during imaging, the operator can know whether or not a radiographic image (reference position image) has been captured at the reference position. As a result, it is possible to reduce the burden on an operator such as an imaging technician at the time of imaging, and it is also possible to shorten the imaging time.

In addition, since the radiographic imaging apparatus 10 provides a notification at the reference position, the reproducibility of imaging of the reference position image is improved when imaging is performed again.

In addition, the reference position image can be reliably captured by the radiographic imaging apparatus 10 having the above-described configuration, and as a result, a doctor or the like can make a more accurate diagnosis based on the reference position image.

In addition, since the reference position image can be reliably captured, it is not necessary to perform re-imaging, the burden on the patient side can be reduced, and the exposure dose can be reduced.

The above-described embodiments are merely examples for implementing the present invention, and the technical scope of the present invention should not be interpreted in a limited manner by these embodiments. That is, the present invention can be implemented in various forms without departing from the spirit or main features thereof.

For example, the radiographic imaging apparatus 10 and the radiographic imaging control apparatus 20 may be mounted on a movable cart or the like and configured as a medical cart. In such a case, the radiographic imaging apparatus 10 and the radiographic imaging control apparatus 20 may be configured to be wirelessly communicable with the radiographic image analysis apparatus 30, the image management apparatus 40, and the client terminal 50.

In addition, the reference position for the target part may be a predetermined reference position as illustrated in FIG. 6, but may be a reference position set at the time of previous imaging, or the reference position may be set according to a disease or the condition of a patient. In this case, the reference position is not limited to being based on the imaging order information, the operator may input or specify the reference position using the operation section 17 or the like.

Further, in the above-described embodiment, the reference position is determined and the reference position image is identified during imaging, but the reference position may be determined and the reference position image may be identified after imaging. In this case, although the imaging controller 11 of the radiographic imaging apparatus 10 includes the identification section 115 and the notifier 116 in the above embodiment, the controller 21 of the radiographic imaging control apparatus 20 may include the identification section 115 and the notifier 116.

Although embodiments of the present invention have been described and illustrated in detail, the disclosed embodiments are made for purpose of illustration and example only and not limitation. The scope of the present invention should be interpreted by terms of the appended claims.

What is claimed is:

1. A radiographic imaging apparatus, comprising:
an imager that captures a radiographic image of a target part of a subject during a predetermined motion on a frame-by-frame basis;
at least one hardware processor that determines, on the frame-by-frame basis, whether or not a skeleton of the target part is at a reference position for the captured radiographic image, and identifies, as a reference position image, the radiographic image when the skeleton of the target part is at the reference position; and
a notifier that provides a notification based on whether or not the reference position image has been identified.

2. The radiographic imaging apparatus according to claim 1, wherein
the at least one hardware processor includes an image analyzer that performs image analysis on the radiographic image on the frame-by-frame basis and identifies the reference position image based on an analysis result of the image analyzer.

3. The radiographic imaging apparatus according to claim 1, wherein
the notifier provides, during the predetermined motion, the notification by outputting a sound when the skeleton of the target part is at the reference position.

4. The radiographic imaging apparatus according to claim 3, wherein
the notifier increases, during the predetermined motion, a volume of the sound as the skeleton of the target part approaches the reference position.

5. The radiographic imaging apparatus according to claim 1, further comprising a display including a display screen, wherein
the notifier displays, during the predetermined motion, information indicating that the skeleton of the target part has reached the reference position on the display screen, when the skeleton of the target part is at the reference position.

6. A radiographic imaging assistance method by a radiographic imaging apparatus, comprising:
capturing a radiographic image of a target part of a subject during a predetermined motion on a frame-by-frame basis;
determining, on the frame-by-frame basis, whether or not a skeleton of the target part is at a reference position for the captured radiographic image, and identifying, as a reference position image, the radiographic image when the skeleton of the target part is at the reference position; and
providing a notification based on whether or not the reference position image has been identified.

7. A non-transitory computer-readable recording medium storing a radiographic imaging assistance program that causes a computer of a radiographic imaging apparatus to perform:
capturing a radiographic image of a target part of a subject during a predetermined motion on a frame-by-frame basis;
determining, on the frame-by-frame basis, whether or not a skeleton of the target part is at a reference position for the captured radiographic image and identifying, as a reference position image, the radiographic image when the skeleton of the target part is at the reference position; and
providing a notification based on whether or not the reference position image has been identified.
